(19) United States
(12) Patent Application Publication
(10) Pub. No.: US 2025/0095097 A1
(43) Pub. Date: Mar. 20, 2025

(54) Evacuation Simulation Module

(71) Applicants: BOWEN KYLE, Nevada City, CA (US); Leo Zliman, Nevada City, CA (US); Igor Zlimen, Nevada City, CA (US)

(72) Inventors: BOWEN KYLE, San Francisco, CA (US); Leo Zlimen, Nevada City, CA (US); Igor Zlimen, Nevada City, CA (US)

(73) Assignee: Ladris Technologies, Inc., Nevada City, CA (US)

(21) Appl. No.: 18/905,589

(22) Filed: Oct. 3, 2024

Related U.S. Application Data
(60) Provisional application No. 63/524,339, filed on Jun. 30, 2023.

(51) Int. Cl. G06Q 50/26; G01C 21/34

(52) U.S. Cl. CPC G06Q 50/265; G01C 21/3461; G01C 21/3492

(57) ABSTRACT

Technology is disclosed for systems and methods for an Evacuation Simulation module generates vehicle trip time and time-series road congestion metrics for vehicles in hypothetical evacuation scenarios. The outputs of the module can be used to understand total clearance time for a neighborhood or region, choke points and areas of worst congestion, individual vehicle routes and trip times, and effects of scenario constraints and mitigation efforts such as road closures, notification urgency, evacuation sequencing, and contraflow. The module contains a large number of input parameters and utilizes high granularity data to model a wide range of specific evacuation scenarios. The model utilizes a novel approach to calculating traffic congestion and a novel application of AI methodology to incorporate human input into the workings of the model.

### CLAIM OF PRIORITY

This application currently claims priority to, and incorporates by reference, prior U.S. Provisional Utility Patent Application No. 63/542,339 filed Oct. 4, 2023.

### TECHNICAL FIELD

Various embodiments relate to technology for simulating evacuation scenarios for fire and other emergency zones.

### BACKGROUND

With global climate change and the current challenges of urban sprawl, agencies like the National Fire Protection Agency, CAL FIRE (The California Department of Forestry and Fire Protection) and City and County Offices of Emergency Management require better technology to simulate and deliver evacuation models depending on a host of ever-changing factors and parameters. While various tools around evacuation and fire department readiness have tried to address the problem, none have heretofore provided an effective, multi-faceted evacuation simulation module.

What is needed therefore is a technical simulation system which calculates traffic congestion, clearance times, and choke points while incorporating human input in re AI methodology, road closures, sequencing, and detailed infrastructure.

### SUMMARY

Technology is disclosed for systems and methods for an Evacuation Simulation module that generates vehicle trip time and time-series road congestion metrics for vehicles in hypothetical evacuation scenarios. The outputs of the module can be used to understand total clearance time for a neighborhood or region, choke points and areas of worst congestion, individual vehicle routes and trip times, and effects of scenario constraints and mitigation efforts such as road closures, notification urgency, evacuation sequencing, infrastructure developments, and contraflow. The module contains a large number of input parameters and utilizes high granularity data to model a wide range of specific evacuation scenarios. The model utilizes a novel approach to calculating traffic congestion and a novel application of AI methodology to incorporate human input into the workings of the model.

### BRIEF DESCRIPTION OF THE DRAWINGS

FIG. 1 depicts a SIMULATION PIPELINE Flowchart consistent with various embodiments.

FIG. 2 depicts a CONGESTION Flowchart consistent with various embodiments.

FIG. 3 depicts a HUMAN FEEDBACK LOOP Flowchart consistent with various embodiments.

FIG. 4 depicts a screenshot of the evacuation simulation product, consistent with various embodiments.

FIG. 5 depicts an advanced embodiment of the evacuation simulation product suitable for use by advanced operators, consistent with various embodiments.

FIG. 6 depicts a screen of the operator platform, consistent with various embodiments.

### DEFINITIONS

A. granularity=refers to the level of detail or the degree of refinement at which the model can represent or process information. It relates to how finely or coarsely the model can analyze, categorize, or manipulate data.

B. edge weights=refer to numerical values assigned to the edges (connections) between nodes (entities) in a mathematical graph (network). These numerical values represent a measure of the strength, distance, cost, or some other relevant attribute associated with the connection between the nodes. In this invention, a mathematical graph is used to represent the road network used in the evacuation simulation. The edges of that graph represent road segments. The edge weights represent the travel time (in seconds) along the road segment. These edge weights are re-calculated at every timestep (via the congestion calculation sub process) to represent the travel time along the segment with evacuation traffic.

### DETAILED DESCRIPTION

The patent or application file contains at least one drawing executed in color. Copies of this patent or patent application publication with color drawing(s) will be provided by the Office upon request and payment of the necessary fee.

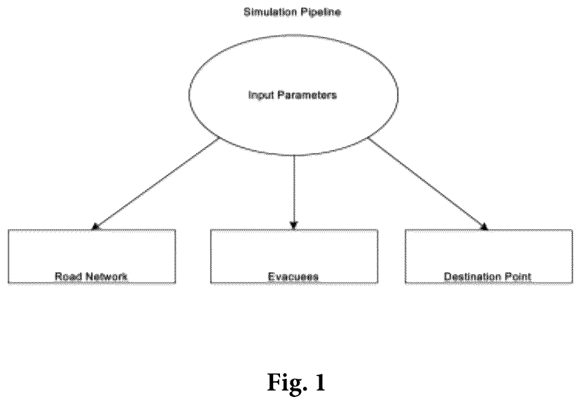

FIG. 1 is a flow diagram of the “Simulation Pipeline.”

The simulation pipeline begins with the creation of a data object to represent evacuees. This object will be used to store data on each individual vehicle simulated in the evacuation scenario. The object consists of containers for storing the vehicle departure time, destination choice, and route.

To determine the vehicle route, and the evolving traffic congestion situation, the container of all evacuees is passed to the routing algorithm to be simulated one at a time to determine the optimal route to the destination for each evacuee. The routing algorithm creates a weighted, directional, graph to represent the road network where the edges are road segments, nodes are road intersections, and edge weights are travel times for the road segment.

The evacuees are simulated one at a time. To simulate the first evacuee, the A* algorithm is used to determine the shortest path between the evacuee's starting location (the starting location is the start node of the nearest edge on the graph to the evacuee's geographical starting location), and each destination point. The destination point with the shortest travel time is selected, and the route to that destination is selected.

Both data objects are stored in the evacuee object. Before the next vehicle can be simulated, the edge weights need to be updated to represent the current traffic state. (See the next section for the mechanics of the calculation congestion).

After the congestion has been re-calculated, the next vehicle is simulated using the same A* method as before. The traffic is re-calculated, and the process repeats.

After each vehicle has been assigned a destination point and route, the travel time for each vehicle is then calculated. To calculate the travel time for an evacuee, the travel time of each leg-segment of the evacuee's trip is calculated based on the congestion mechanic, and the times for each leg are summed to produce a total evacuation time.

From here, statistics are created and aggregated for presentation to the end user.

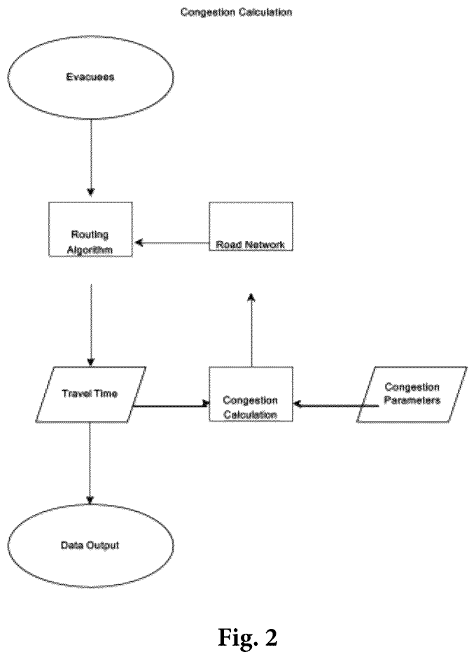

FIG. 2 is a flow diagram mapping the System's Congestion Calculation.

### Congestion Calculation

Congestion on a road segment is calculated for a particular point in time by relating the following parameters: length, num_lanes, num_casr, car_length, vehicle_seperation_time.

wherein length represents the length of the road in meters, num_lanes represents the number of lanes on the road segment, num_cars represents the number of cars on the road at the given point in time, car length represents the average vehicle length in meters, and vehicle separation time represents the minimum possible time (in seconds) between when two consecutive vehicles would pass a stationary object on the roadside.

### Human AI Feedback Loop

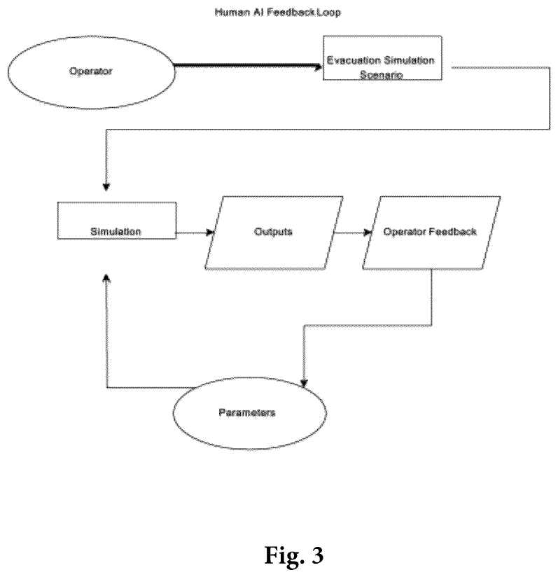

### FIG. 3. Human AI Feedback Loop

The loop of FIG. 3's flow diagram describes how human input can be used to update parameter weights in the model using AI methodologies.

In the first step of the loop, the human operator creates an evacuation scenario by specifying a set of parameters. Then the simulation pipeline is run, and an output is generated.

The operator, having received the simulation output, provides feedback to the mechanism by saying how much (in terms of hours and minutes, positive or negative) they believe the outputs were incorrect by. After receiving a sufficiently large dataset of feedback, across different scenarios, the model can be trained.

The model training occurs by attempting to fit the model parameters for the travel time calculation (congestion calculation) such that the resulting outputs from the simulation minimize the total loss (or error) when compared against the feedback dataset.

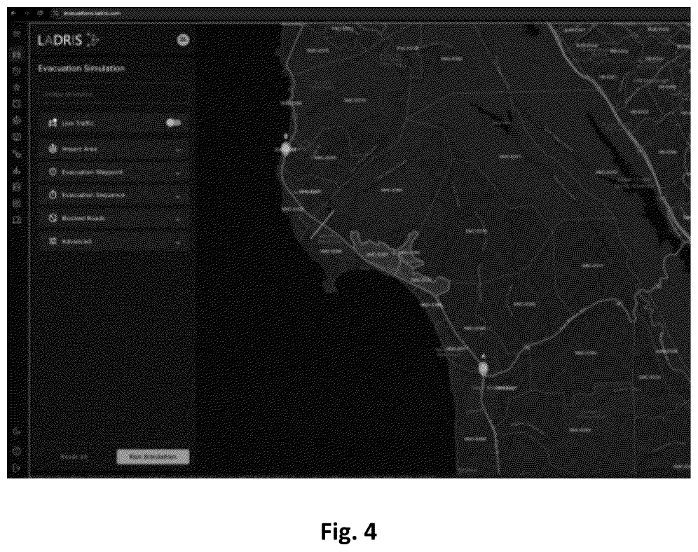

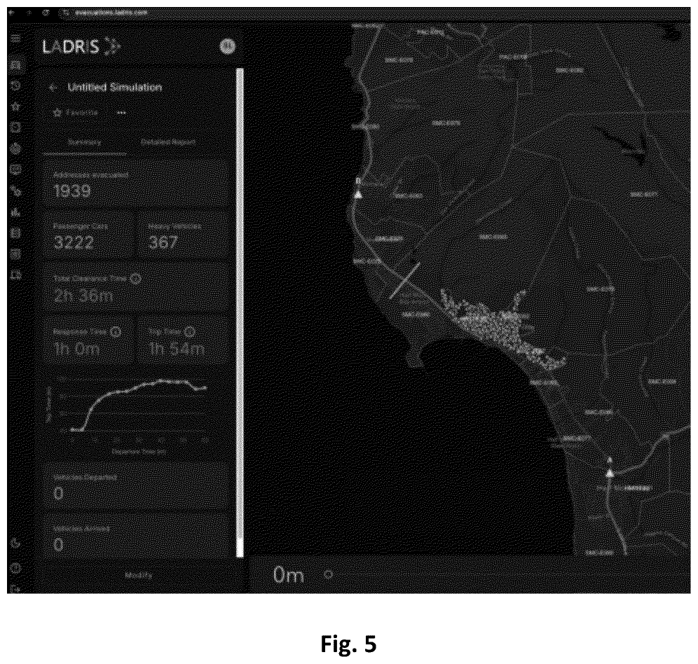

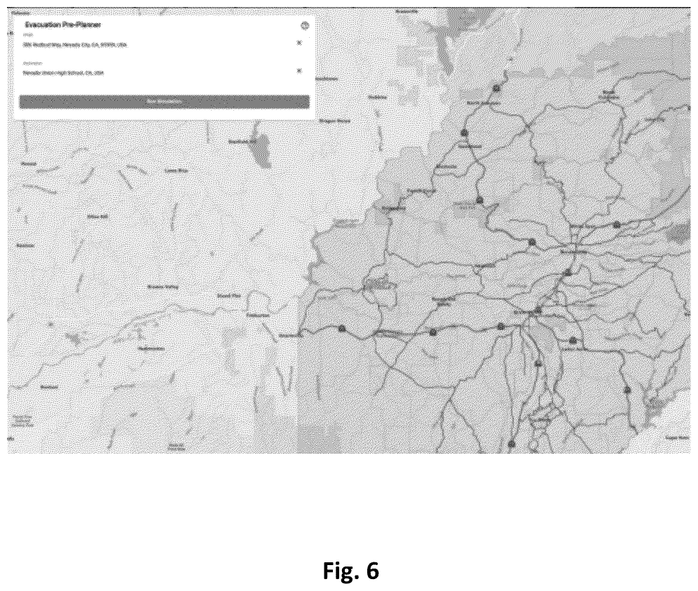

FIG. 4 is a GUI screenshot illustrating the public facing evacuation simulation product. It is a simplified interface compared to the Operator product (shown in, inter alia, FIGS. 5 and 6), but uses the same backend. The exemplary screenshot illustrates the scenario configuration screen in which the user is being prompted to input two addresses, one for the evacuation impact area, the second for the evacuation destination or staging point.

By bounding the evacuation to a particular neighborhood and destination, the backend is then able to use pre-chosen values for all the other simulation parameters and run the simulation.

The prechosen values are, in the embodiment shown in FIG. 4, (inter alia 2 cars per address, 1 mile evacuation radius, 0.2 heavy vehicle percentage, 30 minute departure timeframe)

By so Bounding, the Backend is Able to Produce a Realistic Simulation with Little User Input

This scenario configuration is important because it demonstrates the utility of the invention, and example of usage, for a member of the public. It allows anyone in, e.g., Nevada County to view the potential evacuation times and routes for their neighborhood.

The backend herein functions as follows: the invention produces a simplified user interface around the core evacuation modeling technology. The modeling technology takes the two input parameters from the end user, adds default values for a set of other parameters, and runs the simulation.

The simulation works via the mechanics described supra and infra, by loading road network data and address data, applying network constraints such as drivable roads, lane count, contraflow, and road blocks, calculating vehicle routes vehicle by vehicle, updating congestion and producing statistics (the key stat being evacuation trip time) so that the sim illuminates key insights into the dynamics of the evacuation scenario in, inter alia, in the following way: by
predicting evacuation routes for each vehicle,
trip time for each vehicle,
congestion metrics for each road segment at numerous points in time, and
highlighting bottlenecks and worst chokepoints.

The congestion subprocess is nuanced by incorporating data (inter alia feedback on vehicle trip times specific to the scenario) from end user feedback.

FIG. 5: This is an exemplary screenshot of the operator evacuation simulation product.

It is the more robust and advanced version, but uses much of the same backend tech as the public version. The screen shown in the screenshot is again the scenario configuration screen.

This time, in this embodiment, the user has many more options such as specifying a geographical impact area using zones, polygons or a circle, adding roadblocks, sequencing the evacuation, modifying road network or address data, modifying population numbers geographically and more.

This embodiment of the scenario configuration is important because it demonstrates the utility of the invention, and example of usage, for, e.g., an Emergency manager, firefighter, Sheriff, Traffic Engineer, or consultant. It demonstrates the wide range of parameters incorporated by the modeling technology and therefore, the fidelity of the simulation.

This embodiment illustrates a more advanced user interface around the core evacuation modeling technology described in FIG. 4. This version discloses many of the modeling parameters, e.g.
impacted area,
destination points,
departure timeframe,
evacuation sequencing,
blocked roads,
contraflow
to the end user in order to give them a high level of control over the simulation, and the technical key in giving this control is the ability for the model to respond to a wide range of value choices for each parameter [xyz setting/config/input/output/technology].

FIG. 6: This is the simulation results screen of the operator platform. This portion of the user interface is a visualization of some of the output data generated by the evacuation simulation. On the map, the pink points represent addresses, the colored polylines are road segments. The road segments are colored based on the projected congestion level on that particular segment of road in the simulation. Green is free flow speed, red is heavily congested. On the panel on the left side, we see some basic statistics from the simulation such as “worst case single vehicle trip time” and “average vehicle trip time.”

This is important because it demonstrates the utility of the simulation outputs. The visual example and hard numbers behind evacuation scenarios, such as xyz, are of utmost importance to emergency managers, firefighters, sheriffs, analysts, etc.

This Figure exposes some of the most crucial data outputs (which are, inter alia,
worst case single vehicle trip time,
median case single vehicle trip time, and
road network congestion over time
from our simulation and visually presenting them for end user consumption.

Technically, the key to visually presenting is the generation of granular data including each vehicle route, each vehicle trip time, and road segment by road segment congestion counts, appropriately aggregating this information for the end user and displaying it via map based animation and 2D data visualizations such as histograms or Cartesian charts. The scenario modeling technology was designed to produce these kinds of geospatial outputs.

### Generally

In the Specifications and in this Detailed Description, and the Claims below, and in the accompanying drawings, reference is made to particular features of various embodiments of the invention. It is to be understood that the disclosure of embodiments of the invention in this specification includes all possible combinations of such particular features. For example, where a particular feature is disclosed in the context of a particular aspect or embodiment of the invention, or a particular claim, that feature can also be used—to the extent possible—in combination with and/or in the context of other particular aspects and embodiments of the invention, and in the invention generally.

While multiple embodiments are disclosed, still other embodiments of the present invention will become apparent to those skilled in the art from this detailed description. The invention is capable of myriad modifications in various obvious aspects, all without departing from the spirit and scope of the present invention. Accordingly, the drawings and descriptions are to be regarded as illustrative in nature and not restrictive.

It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments.

In the present disclosure, various features may be described as being optional, for example, through the use of the verb “may;”, or, through the use of any of the phrases: “in some embodiments,” “in some implementations,” “in some designs,” “in various embodiments,” “in various implementations,”, “in various designs,” “in an illustrative example,” or “for example;” or, through the use of parentheses. For the sake of brevity and legibility, the present disclosure does not explicitly recite each and every permutation that may be obtained by choosing from the set of optional features. However, the present disclosure is to be interpreted as explicitly disclosing all such permutations. For example, a system described as having three optional features may be embodied in seven different ways, namely with just one of the three possible features, with any two of the three possible features or with all three of the three possible features.

In various embodiments, elements described herein as coupled or connected may have an effectual relationship realizable by a direct connection or indirectly with one or more other intervening elements.

In the present disclosure, the term “any” may be understood as designating any number of the respective elements, i.e. as designating one, at least one, at least two, each or all of the respective elements. Similarly, the term “any” may be understood as designating any collection(s) of the respective elements, i.e. as designating one or more collections of the respective elements, a collection comprising one, at least one, at least two, each or all of the respective elements. The respective collections need not comprise the same number of elements.

While various embodiments of the present invention have been disclosed and described in detail herein, it will be apparent to those skilled in the art that various changes may be made to the configuration, operation and form of the invention without departing from the spirit and scope thereof. In particular, it is noted that the respective features of embodiments of the invention, even those disclosed solely in combination with other features of embodiments of the invention, may be combined in any configuration excepting those readily apparent to the person skilled in the art as nonsensical. Likewise, use of the singular and plural is solely for the sake of illustration and is not to be interpreted as limiting.

In the present disclosure, all embodiments where “comprising” is used may have as alternatives “consisting essentially of,” or “consisting of.” In the present disclosure, any method or apparatus embodiment may be devoid of one or more process steps or components. In the present disclosure, embodiments employing negative limitations are expressly disclosed and considered a part of this disclosure.

Certain terminology and derivations thereof may be used in the present disclosure for convenience in reference only and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

The term “comprises” and grammatical equivalents thereof are used herein to mean that other components, ingredients, steps, among others, are optionally present. For example, an embodiment “comprising” (or “which comprises”) components A, B and C can consist of (i.e., contain only) components A, B and C, or can contain not only components A, B, and C but also contain one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example, “at least 1” means 1 or more than 1. The term “at most” followed by a number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose limit is the second number. For example, 25 to 100 mm means a range whose lower limit is 25 mm and upper limit is 100 mm.

Many suitable methods and corresponding materials to make each of the individual parts of embodiment apparatus are known in the art. According to an embodiment of the present invention, one or more of the parts may be formed by machining, 3D printing (also known as “additive” manufacturing), CNC machined parts (also known as “subtractive” manufacturing), and injection molding, as will be apparent to a person of ordinary skill in the art. Metals, wood, thermoplastic and thermosetting polymers, resins and elastomers as may be described herein-above may be used. Many suitable materials are known and available and can be selected and mixed depending on desired strength and flexibility, preferred manufacturing method and particular use, as will be apparent to a person of ordinary skill in the art.

Any element in a claim herein that does not explicitly state “means for” performing a specified function, or “step for” performing a specific function, is not to be interpreted as a “means” or “step” clause as specified in 35 U.S.C. § 112 (f). Specifically, any use of “step of” in the claims herein is not intended to invoke the provisions of 35 U.S.C. § 112 (f). Elements recited in means-plus-function format are intended to be construed in accordance with 35 U.S.C. § 112 (f).

Recitation in a claim of the term “first” with respect to a feature or element does not necessarily imply the existence of a second or additional such feature or element.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim in this or any application claiming priority to this application require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

According to an embodiment of the present invention, the system and method may be accomplished through the use of one or more computing devices. One of ordinary skill in the art would appreciate that an exemplary system appropriate for use with embodiments in accordance with the present application may generally include one or more of a Central processing Unit (CPU), Random Access Memory (RAM), a storage medium (e.g., hard disk drive, solid state drive, flash memory, cloud storage), an operating system (OS), one or more application software, a display element, one or more communications means, or one or more input/output devices/means. Examples of computing devices usable with embodiments of the present invention include, but are not limited to, proprietary computing devices, personal computers, mobile computing devices, tablet PCs, mini-PCs, servers or any combination thereof. The term computing device may also describe two or more computing devices communicatively linked in a manner as to distribute and share one or more resources, such as clustered computing devices and server banks/farms. One of ordinary skill in the art would understand that any number of computing devices could be used, and embodiments of the present invention are contemplated for use with any computing device.

In various embodiments, communications means, data store(s), processor(s), or memory may interact with other components on the computing device, in order to effect the provisioning and display of various functionalities associated with the system and method detailed herein. One of ordinary skill in the art would appreciate that there are numerous configurations that could be utilized with embodiments of the present invention, and embodiments of the present invention are contemplated for use with any appropriate configuration.

According to an embodiment of the present invention, the communications means of the system may be, for instance, any means for communicating data over one or more networks or to one or more peripheral devices attached to the system. Appropriate communications means may include, but are not limited to, circuitry and control systems for providing wireless connections, wired connections, cellular connections, data port connections, Bluetooth connections, or any combination thereof. One of ordinary skill in the art would appreciate that there are numerous communications means that may be utilized with embodiments of the present invention, and embodiments of the present invention are contemplated for use with any communications means.

Throughout this disclosure and elsewhere, block diagrams and flowchart illustrations depict methods, apparatuses (i.e., systems), and computer program products. Each element of the block diagrams and flowchart illustrations, as well as each respective combination of elements in the block diagrams and flowchart illustrations, illustrates a function of the methods, apparatuses, and computer program products. Any and all such functions (“depicted functions”) can be implemented by computer program instructions; by special-purpose, hardware-based computer systems; by combinations of special purpose hardware and computer instructions; by combinations of general-purpose hardware and computer instructions; and so on-any and all of which may be generally referred to herein as a “circuit,” “module,” or “system.”

While the foregoing drawings and description may set forth functional aspects of the disclosed systems, no particular arrangement of software for implementing these functional aspects should be inferred from these descriptions unless explicitly stated or otherwise clear from the context.

Each element in flowchart illustrations may depict a step, or group of steps, of a computer-implemented method. Further, each step may contain one or more sub-steps. For the purpose of illustration, these steps (as well as any and all other steps identified and described above) are presented in order. It will be understood that an embodiment can contain an alternate order of the steps adapted to a particular application of a technique disclosed herein. All such variations and modifications are intended to fall within the scope of this disclosure. The depiction and description of steps in any particular order is not intended to exclude embodiments having the steps in a different order, unless required by a particular application, explicitly stated, or otherwise clear from the context.

Traditionally, a computer program consists of a sequence of computational instructions or program instructions. It will be appreciated that a programmable apparatus (i.e., computing device) can receive such a computer program and, by processing the computational instructions thereof, produce a further technical effect.

A programmable apparatus may include one or more microprocessors, microcontrollers, embedded microcontrollers, programmable digital signal processors, programmable devices, programmable gate arrays, programmable array logic, memory devices, application specific integrated circuits, or the like, which can be suitably employed or configured to process computer program instructions, execute computer logic, store computer data, and so on. Throughout this disclosure and elsewhere a computer can include any and all suitable combinations of at least one general purpose computer, special-purpose computer, programmable data processing apparatus, processor, processor architecture, and so on.

It will be understood that a computer can include a computer-readable storage medium and that this medium may be internal or external, removable and replaceable, or fixed. It will also be understood that a computer can include a Basic Input/Output System (BIOS), firmware, an operating system, a database, or the like that can include, interface with, or support the software and hardware described herein.

Embodiments of the system as described herein are not limited to applications involving conventional computer programs or programmable apparatuses that run them. It is contemplated, for example, that embodiments of the invention as claimed herein could include an optical computer, quantum computer, analog computer, or the like.

Regardless of the type of computer program or computer involved, a computer program can be loaded onto a computer to produce a particular machine that can perform any and all of the depicted functions. This particular machine provides a means for carrying out any and all of the depicted functions.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

Computer program instructions can be stored in a computer-readable memory capable of directing a computer or other programmable data processing apparatus to function in a particular manner. The instructions stored in the computer-readable memory constitute an article of manufacture including computer-readable instructions for implementing any and all of the depicted functions.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

The elements depicted in flowchart illustrations and block diagrams throughout the figures imply logical boundaries between the elements. However, according to software or hardware engineering practices, the depicted elements and the functions thereof may be implemented as parts of a monolithic software structure, as standalone software modules, or as modules that employ external routines, code, services, and so forth, or any combination of these. All such implementations are within the scope of the present disclosure.

Unless explicitly stated or otherwise clear from the context, the verbs “execute” and “process” are used interchangeably to indicate execute, process, interpret, compile, assemble, link, load, any and all combinations of the foregoing, or the like. Therefore, embodiments that execute or process computer program instructions, computer-executable code, or the like can suitably act upon the instructions or code in any and all of the ways just described.

The functions and operations presented herein are not inherently related to any particular computer or other apparatus. Various general-purpose systems may also be used with programs in accordance with the teachings herein, or it may prove convenient to construct more specialized apparatus to perform the required method steps. The required structure for a variety of these systems will be apparent to those of skill in the art, along with equivalent variations. In addition, embodiments of the invention are not described with reference to any particular programming language. It is appreciated that a variety of programming languages may be used to implement the present teachings as described herein, and any references to specific languages are provided for disclosure of enablement and best mode of embodiments of the invention. Embodiments of the invention are well suited to a wide variety of computer network systems over numerous topologies. Within this field, the configuration and management of large networks include storage devices and computers that are communicatively coupled to dissimilar computers and storage devices over a network, such as the Internet.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components.

1. A non-transitory computer readable storage medium carrying one or more sets of
instructions which, when executed by at least one processor, generates a simulation of vehicle trip time
and time-series road congestion metrics for public user consumption,
wherein the method for calculating traffic congestion is accomplished one vehicle at a time,
using a weighted directional graph to represent a road segment along a given route, including intersections,
and wherein the total road segment is broken into smaller segments, separated by nodes being intersections, and
the smaller segments define the calculation for edge weighs being travel time in seconds within each section,
and wherein the method for calculating traffic congestion is accomplished by updating the edge weight as
each vehicle is simulated using the route,
the simulation repeating this process for all the vehicles within a demarcated evacuation area,
wherein the total travel time is produced by calculating the sum of the edge weight calculations for each smaller segment and for each vehicle.

2. An evacuation simulation system, comprising a non-transitory computer-readable storage medium and at least one processor,
running at least one sequence of at least one set of instructions which generates for public user consumption, a displayed road network comprising vehicle trip time and road congestion metrics for vehicles in at least one evacuation scenario,
generating and updating an evacuation trip time, contingent on the shortest path for an evacuee toward a safe destination point, by:
creating a data object representing evacuees, said data objects being containers for storing vehicle departure time, vehicle destination choice, and destination route for each individual vehicle,
wherein the container is passed through a routing algorithm which determines optimal route to the destination for each evacuee, therein
determining a navigation route based on parameter data for a geographic region associated with at least one candidate route, said parameters being the initial default values which are updated to repeat the simulation using human input to reset these default values to reproduce and improve the conclusory evacuation trip time;
therein determining the best route and generating map data based on the evacuation trip time.

3. The invention of claim 2, wherein the system creates a weighted directional graph representing the road network, isolating and calculating road segments and nodes, wherein the nodes are road intersections, and wherein the edge weights are travel times for the road segment.

4. An apparatus for generating the total evacuation time for a designated evacuation zone by
enumerating, processing, displaying and updating navigation routes comprising:
at least one processor,
at least one data memory unit,
at least one set of instructions
configured to
calculate evacuation time and road congestion by at least:
specifying geographic impact areas using zones,
loading road network data and address data,
applying network constraints of at least drivable roads, lane count, contraflow, and road blocks;
the apparatus therein calculating vehicle routes individually and by aggregate, initially calculating vehicle by vehicle
and then updating congestion statistics, the key statistic being evacuation trip time,
such that the system, by predicting evacuation routes for each vehicle,
calculates trip time for each vehicle and calculates congestion metrics for each road segment at sequential points in time,
highlighting bottlenecks and choke points;
then updating the default times and then recalculating the trip times and congestion metrics and choke points using user feedback.

5. The Apparatus of claim 4 which further specifies a geographical impact area
using zones delineated by geographic shapes such as polygons and circles,
factoring in roadblocks to aid the travel time calculation,
wherein the apparatus further sequences the evacuation
and further modifies road network data,
and further modified address data,
and further modifies population numbers via accessed human input,
to calculate, display, and update a total evacuation time.

6. The apparatus of claim 4,
which further calculates and updates a
worst case single vehicle trip time,
a median case single vehicle trip time, and wherein
road network congestion is determined on the level of individual road segments over sequentially progressing periods of time,
and wherein the apparatus then visually displays and updates the trip time calculations for end user consumption.

7. The apparatus of claim 4 which further
defines a geographical impact area by
encapsulating geographical impact zones using polygons and circles by
modeling the parameters of at least:
impacted area, destination points, departure points, departure time-frame, evacuation sequencing, blocked roads, and contraflow,
such that the system responds to the real-time change of these parameters, from human feedback, in its total evacuation time calculation.

8. An Evacuation Simulation module which generates vehicle trip time and time-series road congestion metrics for vehicles in an evacuation zone wherein the module, factoring in road closures, blocked roads, and contraflow,
calculates traffic congestion,
determines total clearance time for at least one geographic region,
identifies road choke points,
identifies geographic regions of worst traffic congestion,
identifies individual vehicle routes,
calculates individual vehicle trip times,
and aggregates and updates these parameters to calculate total evacuation time.

9. A computer-generated module utilizing at least one set of instructions which, when executed by at least one processor, generates a first simulation which
calculates road congestion value for public user consumption,
congestion determined by weighing and calculating at least the parameters of
road length,
average car length,
number of car lanes in a particular road segment,
number of cars on the particular road segment at a first point in time,
and vehicle separation time,
vehicle separation time being the minimum time for two consecutive vehicles to pass a stationary point on the instant road segment,
wherein these parameter default values are reset in at least a second simulation with updated weights,
said updating being the simulation's receiving the number of seconds in which the congestion value was inaccurate compared to a given ideal value as received from human input,
and wherein the simulation process repeats itself a threshold number of times such that the simulation trains itself to minimize the total error by comparing calculated travel time to the feedback dataset.

10. A non-transitory computer readable storage medium following at least one set of instructions which,
when executed by at least one processor,
calculates an evacuation trip time
and
an evacuation simulation in which
two sets of addresses are initially assessed, for operator user consumption,
the first address being the evacuation disaster area,
the second address being the evacuation destination,
wherein the simulation predicts an ideal evacuation route based on pre-chosen values for the given evacuation area and the given
evacuation destination,
wherein the prechosen values are at least:
the number of cars per address,
the evacuation radius,
the vehicle density percentage,
and a departure time frame,
and wherein the simulation leverages road network data and address data, and applies the network constraints of drivable roads, lane count, contraflow and road blocks,
and wherein the evacuation simulation then calculates ideal vehicle routes, updating congestion calculations and updating the crucial desired evacuation trip time,
and wherein the simulation repeats itself a threshold number of times such that the simulation trains itself to minimize any evacuation trip time error by comparing calculated travel time to the updated feedback dataset.

11. An evacuation simulation which calculates and filters relevant simulation data for operator user consumption by
toggling live traffic on and off,
highlighting an impact area,
placing red shading and a hard red outline over the area,
allowing user to select a waypoint,
displaying evacuation sequence information,
displaying blocked roads,
placing a yellow line across the route,
said simulation offering the option to reset the filters and offering the option to run simulation,
and wherein
once a “reset all” command is selected, the filters are returned to their default mode, and
once a “run simulation” command is selected, a detailed map is displayed with
a starting and ending point for the simulated route representing the
optimal total evacuation time.

12. A simulation module that processes, updates and displays calculated evacuation simulation data to determine best routes and total evacuation time for operator user consumption,
comprising options to analyze the relevant simulation by:
assessing, weighing and factoring in a calculated number of addresses within a chosen evacuation area which
is represented in purple circles,
assessing, weighing and factoring in a calculated number of passenger cars within the evacuation area,
assessing, weighing and factoring in a calculated number of heavy vehicles within the evacuation area,
assessing, weighing and factoring in a calculated total clearance time for the evacuation area for these
vehicles to reach a waypoint,
assessing, weighing and factoring in a calculated trip time for the requested route from vehicles start location
to the waypoint,
assessing, weighing and factoring in trip time in minutes for a requested route based on departure time in
minutes,
assessing, weighing and factoring in a calculated number of vehicles that have departed along the generated
route,
assessing, weighing and factoring in a calculated number of vehicles that have arrived at the end of the
generated route,
the module further offering an option to modify the route in reaching its total evacuation time determination.

13. The simulation module of claim 12 further featuring an
X-Y axis graph wherein a first colored line represents trip time on the X-axis
and wherein a second colored line on the Y-axis represents departure time,
the axis therein showing the trend of vehicles departing later in the evacuation taking longer to reach their destination.

14. A simulation module that processes, updates and displays calculated evacuation simulation data to determine best routes and total evacuation time,
the module visually displaying routes within a chosen origin starting point and a chosen destination ending point,
the module displaying a publicly-available map displaying the common geographic landmarks of established roads, rivers, bodies of water and landmarks,
the simulation module further offering the option to prompt a “Run Simulation” command
once an origin starting point and a destination ending point are chosen.

15. A non-transitory computer readable storage medium carrying one or more set of instructions which, when executed by at least one processor, produces an evacuation simulation with administrative privileges available to special administrative users comprising emergency
managers, firefighters, sheriff department staff, traffic engineers and staff and consultants and similarly predesignated administrative users,
wherein the simulation, for these administrative users, displays options exclusive to these special administrative, options comprising:
an administrative user may define a geographical impact area using zones, polygons or a circle;
an administrative user may add roadblocks, sequence the evacuation, modify road network or address data, and modify population numbers;
and wherein the simulation, for administrative users, provides a specialized visualization based on the generated evacuation simulation,
wherein the simulation generates a first color for colored-points representing addresses, and wherein the simulation generates a second color for colored-polylines representing road segments,
and wherein the road segments are shown with a third color for projected congestion levels on a particular road segment, such that
a road then shows
green as free flow speed, and
red as heavily congested,
and wherein
the simulation for administrative users further provides special statistics based on the simulation, these special statistics being at least
worst case single vehicle trip time, and
average vehicle trip time,
in determining, displaying and updating the total evacuation time.

16. A simulation module that processes, updates and displays calculated evacuation simulation data to determine best routes and total evacuation time,
wherein the simulation module
toggles on and off live traffic, highlights impact area, places red shading and a hard red outline over the impact area,
identifies an evacuation waypoint,
displays evacuation sequence information,
displays blocked roads by placing a yellow line across the route,
the simulation further providing the option to reset these filters, further providing the
option to run the simulation in a sequence wherein,
once a command “reset all” is selected, the filters are returned to their default mode, and such that
once a command “run simulation” is selected, a detailed map is displayed with a starting and an ending point for the simulated route.

17. The simulation module of claim 16 wherein the default values comprise at least:
two cars per address,
a one-mile evac radius,
a 0.2 heavy vehicle percentage defining the density of traffic, and
a thirty-minute departure timeframe.

18. A simulation module that processes, updates and displays calculated evacuation simulation data to determine best routes and total evacuation time,
wherein the simulation is:
assessing, weighing and factoring in a calculated number of addresses within a named evacuation area,
assessing, weighing and factoring in a calculated number of passenger cars within a named evacuation area,
assessing, weighing and factoring in a calculated number of heavy trucks within a named evacuation area,
assessing, weighing and factoring in a calculated total clearance time for a named evacuation area,
assessing, weighing and factoring in a calculated response time for a named evacuation area,
assessing, weighing and factoring in a calculated trip time for requested route,
assessing, weighing and factoring in a geographical visual representation of trip time for a requested route in minutes
based on departure time in minutes,
and wherein the module is additionally
assessing, weighing and factoring in a calculated number of vehicles that have departed along the generated
route, and
further assessing, weighing and factoring in a calculated number of vehicles that have arrived at the end of the
generated route, and
further offering the option to modify the route.

19. A simulation module that processes, updates and displays calculated evacuation simulation data to determine best routes and total evacuation time,
wherein the simulation comprises
a map comprising routes within a named origin” starting point and
a named destination ending point, and
wherein the map displays common geographic landmarks comprising at least
established roads, rivers, bodies of water, and landmarks,
the simulation further comprising the option to follow a “Run Simulation” prompt once an origin starting point and a destination
ending point are demarcated.
